(19) United States
(12) Patent Application Publication
(10) Pub. No.: US 2005/0197625 A1
(43) Pub. Date: Sep. 8, 2005

(54) Device for the dosed discharging of a liquid agent and infusion pump

(76) Inventors: Ulrich Haueter, Grosshoechstetten (CH); Erich Imhof, Utzenstorf (CH); Urs Kipfer, Luetzelflueh (CH)

Correspondence Address: DORSEY & WHITNEY LLP;INTELLECTUAL PROPERTY DEPARTMENT, 50 SOUTH SIXTH STREET, MINNEAPOLIS, MN, 55402-1498, US

(21) Appl. No.: 11/067,471

(22) Filed: Feb. 25, 2005

Related U.S. Application Data
(63) Continuation of application No. PCT/EP03/09546, filed on Aug. 28, 2003.

(30) Foreign Application Priority Data
Aug. 30, 2002 (DE) 102 40 165.9

(51) Int. Cl. A61M 37/00

(52) U.S. Cl. 604/131

(57) ABSTRACT

A device for delivering a liquid agent in doses, the device including a receptacle for storing the liquid agent and a propulsion mechanism for advancing a piston toward an outlet of the receptacle in order to expel a dose of the agent, wherein the propulsion mechanism permanently acts on the piston with a propulsive force, and wherein the device further includes a mechanism for releasably blocking a piston advance. The invention encompasses embodiments in the form of an infusion pump adapted to include the device for delivering and a controller for controlling at least the mechanism for releasably blocking.

### CROSS REFERENCE TO RELATED APPLICATION(S)

This application is a Continuation of International Patent Application No. PCT/EP2003/009546, filed on Aug. 28, 2003, which claims priority to German Application No. 102 40 165.9, filed on Aug. 30, 2002, the contents of which are incorporated by reference in their entirety herein.

### BACKGROUND

The present invention relates to a devices and methods for delivering or administering a substance. More particularly, it relates to injection and infusion devices, and to methods of making and using them. Even more particularly, it relates to devices, such as infusion pumps, adapted to provide for the long-term, generally uniform, periodic release of a small amount or dose of a medicinal agent.

In the medicating of patients, it is often advantageous to provide the patients with small doses of a medical agent over a comparatively long period of time instead of injecting the patients with comparatively large doses of a medicine at relatively few time points. For example, in the treatment of diabetes, it is advantageous to supply the required agent, namely insulin, in microdoses in a quasi-continuous manner over a longer period of time. This opens up the possibility of optimally adjusting the blood sugar of the patient. Recent years have seen the increasing development of diagnostic systems that make possible a quasi-continuous monitoring of medically relevant parameters, for example blood sugar. In cooperation with a quasi-continuous microdosing, an optimal medicating can thus be achieved.

Available for this purpose are infusion pumps that include a reservoir for the liquid agent to be administered and an administration mechanism, for example a microdosing pump. Infusion pumps are known to those skilled in the art, and representative examples include the pumps disclosed in U.S. Pat. Nos. 4,969,874 and 6,053,893, the disclosures and teachings of which are incorporated herein by reference.

Typically, in order that infusion pumps may be carried on or by patients, the pumps have their own energy supply. Usually the reservoir is designed as an ampoule having a piston or plug that is propelled in the direction of an ampoule opening in order to expel the agent. The service life of the energy supply here depends mainly on the frictional forces prevalent in the reservoir. However, the goal is that the energy supply be as long-lived as possible, so that at any time the patient can depend on the fact that the agent so important to him will actually be administered. As is well known, the recharging of storage batteries is complicated, time consuming, and can be forgotten. If disposable batteries are used as the energy supply, the replacement of the batteries usually requires an opening of the infusion pump. However, for hygienic reasons the pump should be hermetically sealed to the degree possible, so that a battery replacement is disadvantageous.

### SUMMARY

An object of the present invention is to provide a device for the dosed discharging of a liquid agent, which device is operable in an energy-saving manner and is simple and reliable to operate. In addition, in some embodiments, an infusion pump with such a device is provided.

In one embodiment, the present invention comprises a controller for controlling the delivery of a liquid agent in doses, comprising a propulsion mechanism for moving a piston to deliver a dose of the agent, wherein the propulsion mechanism is adapted to exert a motive force on the piston substantially constantly unless interrupted, and a mechanism for selectively interrupting the motive force. In some embodiments, the controller is operably incorporated into an infusion pump and, in some embodiments, the controller further comprises an electronic component operably coupled to the mechanism for selectively interrupting the motive force.

In one embodiment, the present invention comprises an infusion pump for the long-term release of an agent, comprising a receptacle for storing the liquid agent, a propulsion mechanism for advancing a piston toward an outlet of the receptacle in order to expel the agent, wherein the propulsion mechanism continuously acts on the piston with a propulsive force, a blocking mechanism for blocking and allowing the advance of the piston, and a control apparatus that repeatedly releases the blocking mechanism in order to expel the agent in repeated, selected doses. In some embodiments, the control apparatus is electronic and/or digital, and may comprise suitable electronic components, include suitable chips or microprocessors.

A device according to the present invention includes a receptacle for the storage of a liquid agent and a propulsion mechanism for propelling a piston toward an outlet of the receptacle in order to expel the agent, wherein the propulsion mechanism acts permanently on the piston with a propulsion force and a blocking mechanism is provided, by means of which a piston advance is permanently blocked and, in order to expel the agent, released in a controlled manner. Advantageously, in order to expel the agent, energy is consumed only to activate the blocking mechanism and not to propel the piston of the receptacle, since, according to the invention, the propulsion mechanism can be prestressed prior to the placing into operation of the device in such a way that doses of the agent can be repeatedly expelled without renewed pre-stressing of the propulsion mechanism. The energy for the pre-stressing of the propulsion mechanism can thus be provided prior to the placing into operation of the device through, for example, an external energy source, an external mechanism, or manual means. Thus, comparatively little energy is required for the operation of the device itself. (As used herein, the term “blocked” is intended to be synonymous with locked, fixed, secured, and the like, i.e., a fixing or securing in a particular state, situation or method of operation, including, as appropriate, a permanent or continuous fixing or securing, or a temporary, releaseable, selective or controllable fixing or securing.)

At the same time, the present invention makes allowances for safety considerations, since the blocking mechanism is designed such that the piston advance is permanently blocked, so that an overdosing is effectively excluded, for example in the case of a failure of the device electronics, etc. According to the invention, the blocking mechanism is released only at the point in time of the expelling of the agent. Preferably, the blocking mechanism is designed in such a way that upon the unblocking of the piston propulsion the piston is driven forward only by a predetermined stroke length and that the blocking mechanism automatically re-blocks after the driving of the piston by the predetermined stroke length. The predetermined stroke length appropriately corresponds to a minimally pre-settable dose. Thus, in this preferred embodiment a long-term release of the agent can be controlled in a simple manner. This is by virtue of the fact that, instead of requiring a presetting each time of a target value for the adjustment of the piston to achieve a desired dosing, in one embodiment, an electronic control performs a count of how often the blocking mechanism has released the propulsion of the piston. With knowledge of the agent dose that is expelled with a single release of the blocking mechanism, in a simple manner the total dose expelled can be calculated through multiplication of the single dose by the number of releases of the blocking mechanism. Thus, according to one exemplary preferred embodiment, a simple temporal control can be used to control the long-tem dosing.

Also advantageous is the fact that the dosing mechanism is virtually always at rest, since it must be activated only at the comparatively few points in time of an expelling of the agent. This helps to save energy.

According to a preferred embodiment, the propulsion mechanism pre-stresses the piston relative to a reference point that is kept fixed in relation to the blocking mechanism. This reference point can therefore be, for example, a housing or a wall of the agent receptacle.

In some embodiments, the present invention may comprise any propulsion mechanism suitable for repeated displacement of a piston, including any such mechanisms involving pre-stressing. For example, the propulsion mechanism can include a compression spring, one end of which acts on the piston and the other end of which is held fixed, for example relative to the housing of the infusion pump. According to the invention, the compression spring is pre-stressed prior to the placing into operation of the device. Upon the release of the blocking mechanism, the compression spring can dissipate the pre-stress in a step by step manner, until a relaxed end position is reached. According to another exemplary embodiment, the propulsion mechanism can be a torsion spring, as the latter is known from other mechanical devices, for example mechanical clocks. For example, the torsion spring can be a wound spiral spring or coil spring of round or rectangular cross section, which spring is accepted in a housing, one end of the torsion spring producing a rotary movement in the propulsion mechanism, which movement is converted in a known manner into a propulsive force for the piston, for example with the aid of gears or through thread engagement of a rotatable, driven threaded bar.

According to another embodiment, the propulsion mechanism can include a pressurized chamber in which a gas is stored under pressure and which displays a flexible chamber wall, so that the gas can expand in order to propel the piston when the blocking mechanism is released. Thus, in this embodiment, prior to the placing into operation of the device the pressurized chamber is pumped up or, as the case may be, a suitable pressurized-gas reservoir is installed in the device, for example a pressurized-gas bottle such as are economically available on the market.

According to the present invention, magnetic forces can also in principle be used for propelling the piston. To this end, sufficiently strong permanent magnets can be provided on the housing of the device and on the piston of the receptacle, such magnets being available economically on the market.

In some preferred embodiments, the blocking mechanism includes a blocking means and a catch that work together, which catch is movable and in a first position blocks the piston advance and in a second position releases the piston advance in order to bring about the expelling of the agent. Appropriately, the displacement of the catch requires comparatively little energy, so that the expelling of the agent can be effected in an energy-saving manner. Appropriately, the displacement movement is designed as a simple back-and-forth movement, for example as the tipping movement or swinging movement of a lever.

In some embodiments, the displacement of the catch compliments or is coordinated with a displacement of another catch such that at all times one catch prevents an uncontrolled advance of the piston beyond the preset stroke length.

According to one preferred embodiment, the blocking mechanism is designed in a manner comparable to an escapement of a mechanical clock movement, which includes a blocking means, for example a ratchet wheel provided with teeth or a piston rod provided with teeth, and a catch that works together with the blocking means in order to block and unblock in a controlled manner a displacement of the blocking means. As used herein “teeth” (or toothing) is intended to mean any suitable type of generally regular projections, textures or structures that engage with a suitable corresponding or complementary projections, textures or structures to transmit force.

Appropriately, the catch is formed as a swingable anchor escape lever with two pawls that engage the teeth of the corresponding blocking means. In this way, through a simple and energy-saving back-and-forth movement of the anchor escape lever, the blocking means is moved along in each case by one tooth of the teeth. According to the present invention, the movement of the blocking means is converted into an advance of the piston, the propulsive force being applied by the propulsion mechanism and not by the blocking means. Preferably, the piston is fixedly connected to the blocking means, so that the piston, due to the permanent application of force, permanently exerts a tractive force on the blocking means, which force is released through a disengagement of a pawl from the teeth, so that the blocking means is displaced until the teeth becomes engaged with the other pawl, which automatically stops the advance of the piston. Thus, the dose to be expelled is predetermined through the teeth, namely through a whole-number multiple of the tooth spacing, in particular one times the tooth spacing of the teeth. Through a moving back of the catch or of the anchor escape lever, as the case may be, the blocking mechanism is moved back into its initial position, in which the piston advance is permanently blocked.

Appropriately, the anchor escape lever of the catch is formed such that during engagement of one catch with the teeth of the blocking means, the other catch is positioned centrally between two teeth of the teeth. If the anchor escape lever is now swung, then the above-described course of movement is triggered.

The teeth that work together with the catch can be provided at an appropriate location in the mechanism, as known from the prior art. According to one embodiment, the teeth are provided on the outer circumference of a ratchet wheel. In this case, a rotary motion of the ratchet wheel is transferred to the propulsion of the piston. Appropriately, in this embodiment, a holding means, for example a cable or band, is wound around a rotational axis or an outer circumference of the ratchet wheel and this holding means is unwound upon the advance of the piston.

According to another embodiment, the teeth are applied to a toothed rack, the displacement of which is transferred directly or indirectly to the piston advance. Advantageously, in this embodiment, a holding means is not included, so that a potential source of failure of the device is eliminated.

According to another embodiment, the blocking mechanism is designed as a spindle blocking, comparable to a spindle escapement known from clock construction. In this embodiment, the catch includes a rotationally-movable shaft that carries two blocking projections that work together with teeth on the top side on opposite sides of the rotary axis of the ratchet wheel, wherein a rotation of the shaft releases the engagement of a blocking projection with the teeth and thus triggers a further turning of the ratchet wheel, until another tooth of the teeth of the ratchet wheel reaches a stopping position with the opposing blocking projection, which automatically ends the piston advance. The blocking teeth can here be provided on a top side of a separate ratchet wheel, in which case a holding means, for example a band or cable, is appropriately wound around the rotary axis or an outer circumference of the ratchet wheel, which holding means restrains the piston. For the piston advance, the holding means is unwound in a controlled manner. Alternatively, the blocking teeth can also be applied to a top side of a separate ratchet wheel, the rotational movement of which is transferred by means of a gearing mechanism to a piston rod or threaded bar or another advancing means. Finally, the blocking teeth can also be arranged directly on a front side of the piston rod or threaded bar, which serves directly or indirectly the advancing of the piston.

Appropriately, the blocking projections of the spindle blocking are arranged in a staggered manner on the rotationally-movable shaft, so that at all times one of the blocking projections blocks an uncontrolled rotation of the ratchet wheel, wherein in first angular position of the shaft a first blocking projection engages the teeth of the blocking means and the second blocking projection is positioned centrally between two teeth, and in a second angular position of the shaft the second, opposite blocking projection engages the teeth of the blocking means and the first blocking projection is positioned between two teeth. Thus, through a simple back-and-forth swinging of the rotationally-movable shaft, the ratchet wheel can be rotated forward by one tooth, the propulsion force being provided through the propulsion mechanism and not through the blocking mechanism.

In some preferred embodiments, for the triggering of the expelling or delivery of the agent, the catch is displaced against a restoring force of a restoring means that attempts to put the catch back into a resting or initial position. Thus, the blocking mechanism automatically returns into a resting or initial position and the only energy required in order to trigger the expelling of the agent is that needed to displace the catch against the restoring force. The releasing of the blocking mechanism can be initiated mechanically, for example by the pressing of a button, if the device is manually operable, or through an actuation means that is triggered by an electronic control of the device.

In some preferred embodiments, the propulsion mechanism can be pre-stressed over the entire stroke of the piston, so that the piston can be advanced until the agent has been completely expelled from the receptacle without requiring the propulsion mechanism to be pre-stressed again. It is advantageous that the pre-stressing work required for this can be performed manually or by an external energy source before the placing into operation of the device, so that the energy source of the device can be completely spared.

According to another embodiment, however, the propulsion mechanism can be pre-stressed over a portion of the maximal piston stroke, so that the propulsion mechanism must be pre-stressed again one or several times up to the complete expelling of the agent. Appropriately provided for this purpose is an indicator device that indicates to the user that the propulsion force provided by the propulsion mechanism has fallen under a pre-settable or preset threshold value. This indicator device can operate mechanically, for example through a mechanical activation of a color field as the indication, or can be triggered electronically, for example in the form of a warning on an LCD display or the like, if necessary aided by an acoustic warning signal, as an indication that the device must be pre-stressed again.

In principle, the mechanical work required for the pre-stressing of the propulsion mechanism can also be performed manually by the user of the device, or through connection to an external energy source. If, however, the propulsion mechanism is not prestressed even after several warnings to the user, then the necessary energy is appropriately provided by the energy source of the device, so that it is ensured that the device is ready for operation at all times.

According to another embodiment, the blocking mechanism can also be designed as a band block, wherein the band block clamps a band that restrains the piston against the permanently active propulsion force of the propulsion mechanism and wherein upon the release of the band block the slipping through of the band is converted into the piston advance. Appropriately, the band block is formed such that this permanently clamps the band, for which purpose suitable measures from the prior art are known to the specialist in this field.

In principle, the band-blocking mechanism can be combined with the above-mentioned anchor escape lever or blocking spindle. In this embodiment, the displacement of the band block and of the anchor escape lever or blocking spindle are appropriately coordinated such that first the anchor escape lever or, as the case may be, the rotationally-movable shaft with the blocking projections is swung from one end position to the other end position. In this, a turning of the ratchet wheel is still blocked by the band block. Through the release of the band block, the ratchet wheel can then be rotated until one tooth of its teeth meets the pawl or the blocking projection. After this, the band block again permanently blocks. For a renewed expelling of the agent, the anchor escape lever or, as the case may be, the shaft with the blocking projections is swung back into the other end position. Thus, the catch need be displaced only once for an expelling of the agent.

Other objects, features and advantages of the present invention may be understood with reference to the accompanying drawings and description, and the appended claims.

### BRIEF DESCRIPTION OF THE DRAWINGS

FIG. 1 depicts an expelling device according to an embodiment of the present invention with an anchor escape lever blocking,

FIG. 2 depicts a variant of the embodiment of FIG. 1 with a blocking spindle,

FIG. 3 depicts another embodiment of the expelling device according to the present invention, wherein a ratchet wheel is attached to an end side of a toothed rack or threaded bar, which ratchet wheel works together with a blocking spindle,

FIG. 4 depicts a variant of the embodiment according to FIG. 4, wherein an anchor escape lever is provided, which works together with the teeth of a toothed rack,

FIG. 5 schematically depicts an expelling device according to another embodiment of the present invention, wherein the piston advance is produced through expansion of a gas reservoir that is pressurized, and

FIG. 6 depicts another embodiment of the expelling device according to the present invention.

### DETAILED DESCRIPTION

In the Figures, identical reference numerals indicate identical or functionally similar components or component groups.

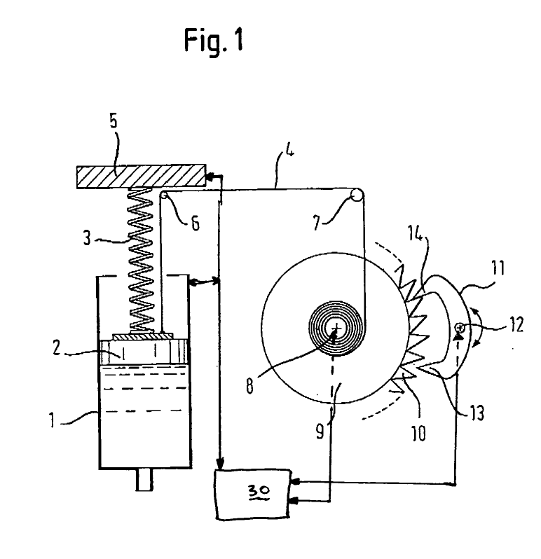

FIG. 1 schematically shows a first embodiment of an expelling device according to the present invention. This is preferably part of an infusion or injection device, for example an infusion pump for long-term medicating of patients or animals, for example for blood-sugar adjustment. As is shown in FIG. 1, the device includes a reservoir 1, in which a liquid containing the agent is stored. The receptacle 1 displays at its front end an opening through which the agent is expelled, for example to a hollow cannula. Situated opposite the expelling opening is the axially displaceable piston 2, the axial adjustment of which in the direction of the expelling opening causes the expelling of the agent. The piston 2 is permanently pre-stressed by means of the compression spring 8 against a fixed reference point 5, which is stationary in relation to the blocking mechanism in the right side of the image and/or in relation to the receptacle 1. Attached to the back side of the piston 2 is a holding means 4, for example a cable or band, which restrains the piston against the restoring force of the spring 3. The holding means 4 is deflected at the deflection points 6 and 7, for example at deflection rollers or deflection pins. The other end of the holding means is connected to the blocking mechanism represented in the right-hand portion of the image.

For this purpose, as shown in FIG. 1, the other end of the holding means 4 is wound onto the ratchet wheel 9, whether this is directly in a circumferential channel of the ratchet wheel 9 or onto a pin arranged on the rotary axis 8 of the ratchet wheel 9. According to FIG. 1, the ratchet wheel 9 displays on its outer circumference a preferably circular ratchet teeth 10 extending in the rotational direction, the teeth of which teeth work together with the pawls 13, 14 of the anchor escape lever 11. The blocking mechanism according to FIG. 1 is overall comparable to an anchor escapement as known from typical clock construction. Thus, the anchor escape lever 11 can be swung back and forth around the axis 12 between a first end position, in which the pawl 14 rests against a tooth of the teeth 10, and a second end position, in which the pawl 13 rests against a tooth of the teeth 10. The anchor escape lever 11 can be restored to one of the end positions by a restoring means (not shown). It is thus ensured that the ratchet wheel 9 cannot rotate forward in an uncontrolled manner.

As is schematically represented in FIG. 1, the anchor escape lever 11 is designed in such a way that when the pawl 14 is engaged with the ratchet teeth 10, the other pawl 13 is positioned centrally between two teeth of the teeth. Upon further rotation of the ratchet wheel 9, the pawl 13 is consequently carried along by a teeth surface and thus the anchor escape lever is tipped back into the other end position.

In order to place the device into operation, the compression spring 3 is prestressed, for example through introduction of a new receptacle with the piston 2 maximally pushed back. In this, the ratchet wheel 9 is turned back until the holding means 4 is tensioned. In order to turn back the ratchet wheel 9, a restoring means (not shown), for example a torsion spring or an electric restoring mechanism, can be provided. For the expelling of the agent, the anchor escape lever 111 is swung from a first end position to the second end position. Thereby, due to the pre-stressing through the compression spring 3, the piston 2 is propelled and an expelling of the agent effected. Simultaneously, the holding means 4 deflected around the deflection grooves 6 and 7 turns the ratchet wheel 9 ahead until one of the pawls 13, 14 of the anchor escape lever comes to rest against a tooth of the ratchet teeth 10. A forward turning of the ratchet wheel 9 is then excluded. The ratchet wheel can be turned forward only after a swinging back of the anchor escape lever 11 into the other end position. Thus, the expelled dose of the agent is preset in a one-to-one manner through the ratchet teeth 10 of the ratchet wheel 9. The angular displacement of the ratchet wheel 9 by one tooth is converted one-to-one into a predefined stroke of the piston 2.

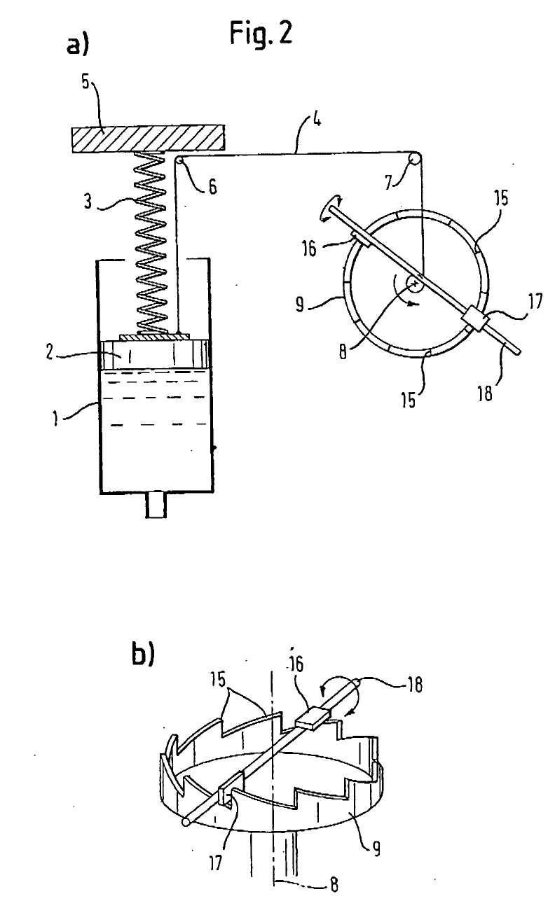

FIG. 2 shows a variant of the embodiment of FIG. 1 in which the blocking mechanism has a design comparable to a blocking spindle known from clock construction. In this variant, teeth 15 are provided on a top side of the ratchet wheel 9, which work together with the two blocking projections 16, 17 of a shaft 18. The shaft 18 is supported such that it is rotatable, around a rotary axis, in a radial manner with respect to the rotary axis 8 of the ratchet wheel 9. The shaft 18 is rotatably supported such that it can be rotated back and forth only between a first end position, wherein the blocking projection 17 lies against one of the teeth of the ratchet teeth 15, and a second end position, wherein the other blocking projection 16 lies against one of the teeth of the ratchet teeth 15, so that one of the two blocking projections 16 and 17 accordingly swings into engagement and the other out of engagement. Thus, it is ensured that the ratchet wheel 9 cannot turn forward in an uncontrolled manner. Rather, the ratchet wheel can always be turned forward by only one tooth, which is brought about by the rotation of the shaft 18 from one end position into the other. In this embodiment too, the other end of the holding means 4 is wound either into a circumferential channel of the ratchet wheel 9 or around a pin placed onto the rotary axis 8 of the ratchet wheel 9 and is unwound upon the advancing of the piston 2.

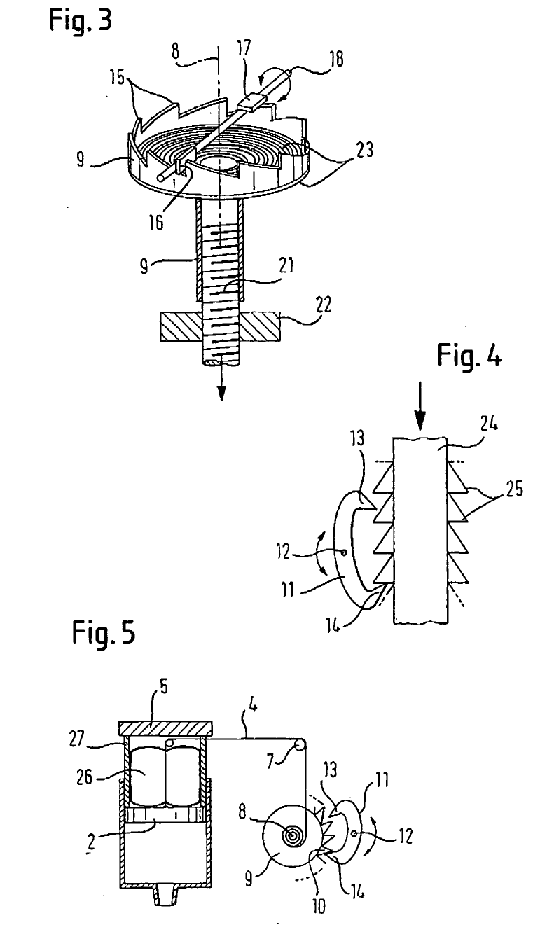

In principle, however, the blocking mechanism can also be integrated into the device such that a holding means is superfluous. FIG. 3 shows a second embodiment of the expelling device according the invention that does not comprise a holding means. According to FIG. 3, the ratchet wheel 9 is attached directly to one end of a threaded bar 21 serving the piston propulsion. Prior to the placing into operation of the device, the ratchet wheel 9 is turned back against the restoring force of a torsion spring 23 or another suitable restoring means. The blocking spindle represented schematically in FIG. 3 then triggers a turning of the ratchet wheel 9, in the manner described above, by one tooth in each case. The outer thread of the threaded bar 21 engages an inner thread, provided on a housing of an infusion pump or on the ampoule 1, of a section 22, so that the rotation of the threaded bar 21 is converted into a piston advance. If the section 22 is fixedly connected to the housing, then the threaded bar can be connected to the ratchet wheel 9 in a rotationally-secured and axially-movable manner and itself form a piston rod. Alternatively, the threaded bar 21 can be fixedly connect to the ratchet wheel. In this case, the section 22 is guided by the housing in an axially-linear manner and can form the piston rod.

It should be understood that the ratchet wheel 9 according to FIG. 3, instead of being provided with a ratchet teeth 15 on the top side, can as well be provided with ratchet teeth on the outer circumference of the ratchet wheel 9, comparable to FIG. 1. As is evident to one skilled in the art, the ratchet wheel 9 can also be connected to the threaded bar 21 serving the piston propulsion via a gearing mechanism, so that the turning movement of the ratchet wheel 9 is transferred to the threaded bar 21 via the gearing mechanism.

FIG. 4 shows a further embodiment of the expelling device according to the present invention that does without a holding means. In this device, serving the piston propulsion is a rod 24 that is designed as a toothed rack with ratchet teeth 25. The ratchet teeth 25 work together with the rotatably-supported anchor escape lever 11, which functions in a manner similar to the anchor escape blocking represented in FIG. 1. Thus, in a first end position of the anchor escape lever 11, the pawl 14 lies against a tooth of the ratchet teeth 25. Through a swinging of the anchor escape lever 11 into the other end position, the pawl 14 becomes disengaged from the ratchet teeth 25 and the other pawl 13 becomes engaged with another tooth of the ratchet teeth 25. Due to the permanently-active propulsion force generated by the propulsion mechanism (not shown), the toothed rack 24 is advanced, in order to effect the expelling of the agent from the receptacle 1. In this, the pawl 13 glides along the profile of a ratchet tooth and is lifted, so that the anchor escape lever 11 is swung back into the initial position, in which the pawl 14 blocks the further propulsion of the piston. Here, the anchor escape lever 11, during the swinging from the first end position shown in FIG. 4 into the other end position (not shown), is swung against the restoring force of a restoring means (not shown), so that the swinging back of the anchor escape lever 11 into the blocking position represented in FIG. 4 is ensured.

FIG. 5 shows another embodiment of the expelling device according to the present invention. According to FIG. 5, the force for the propulsion of the piston is applied by a gas stored in a pressurized chamber 26. Prior to the placing into operation of the device, the pressurized chamber 26, in the rearward end position of the piston 2, is filled with a pressurized gas under high pressure. The pressurized chamber 26 has a flexible wall, so that the gas expands and the pressurized chamber 26 can expand when the blocking mechanism enables the piston advance. As is shown in FIG. 5, the pressurized chamber 26 is supported on a housing section 5 and on the rearward base of the piston 2 and is laterally guided by a sleeve 27, so that the gas can expand the pressurized chamber 26 only in one direction, namely in the piston-propulsion direction, i.e. in the axial direction of the receptacle 1. In principle, the blocking mechanism can be designed according to any of the embodiments described here. In order that the pressurized chamber 26 can expand, at least its side walls must be flexibly formed. The pressurized chamber can, for example, be ring-shaped, so that the holding means 4 can be guided through the pressurized chamber up to the piston 2.

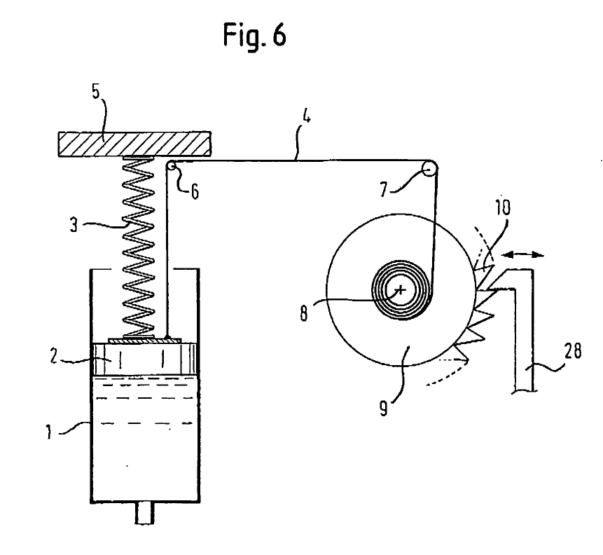

FIG. 6 shows a further embodiment of the expelling device according to the invention. Instead of the above-described anchor escape blocking (cf. FIGS. 1, 4) or blocking spindle (cf. FIGS. 2, 3), the blocking mechanism includes a first catch 28 and a second catch (not shown) substantially identical to the first catch 28. The ratchet wheel 9 displays on its outer periphery teeth 10, which work together with the first catch 28. Further provided is a second set of ratchet teeth (not shown), which work together with the second catch. The second ratchet teeth can be provided on the same or on another ratchet wheel. The two catches can be swung, as indicated by the double arrow, it being ensured, however, that when one catch swings away from the corresponding ratchet teeth, the other catch automatically engages the other corresponding teeth. Thus, it is always ensured that the ratchet wheel cannot spin uncontrolled. Through a coordinated displacement of the catches, the ratchet wheel is thus turned forward by a half tooth. Upon the coordinated swinging back of the two catches, the ratchet wheel is again turned forward by a half tooth and the two catches revert to their initial positions. Upon the turning of the ratchet wheel 9, the other end of the holding means 4 is unwound. Due to the permanently active propulsion force of the compression spring 3, the piston 2 is advanced in order to expel the agent.

According to a further variant of this embodiment, the catch according to FIG. 6 may have a forked shape, with two blocking projections that, according to the angular position of the fork, engage the ratchet teeth 10 either on the left or on the right peripheral edge of the ratchet wheel 9. Here, the spacing between the two blocking projections is dimensioned such that during the swinging of the fork-shaped catch, one of the two blocking projections engages the ratchet teeth at all times.

To operate the expelling device, first the propulsion mechanism is pre-stressed, for example through a pushing back of the compression spring or rotating back of a torsion spring. At the same time, the blocking mechanism is brought into a starting position, in which a pawl or a blocking projection engages the ratchet teeth 10 or 15, as the case may be. Through displacement of the blocking mechanism, for example through a swinging of the anchor escape lever shown in FIG. 1 or 4 or the blocking spindle shown in FIG. 2 or 3, a turning forward of the ratchet wheel 9 is triggered, until a tooth of the ratchet teeth 10, 15 again becomes engaged with a pawl or a blocking projection. In this, the movement of the ratchet wheel or of the piston rod shown in FIG. 4 is converted into the piston propulsion. The propulsive force necessary for this originates from the propulsion mechanism and not from the blocking mechanism. For a renewed or repeated expelling of the agent, the blocking mechanism is actuated again.

In principle, the actuation of the blocking mechanism can occur manually, for example through the pressing of a button on a mechanically-actuated injection apparatus, for example an injection pen for diabetes patients. In some preferred embodiments, however, the expelling device according to the present invention is electronically controlled, for which purpose a suitable electronic control (e.g., a suitable microprocessor, switch, or other suitable electronic components or component), depicted in FIG. 1 at 30 is provided, which control with the aid of an actuation means (not shown) releases the blocking mechanism in a controlled manner. The control or controller 30 may also sense and control other functions, aspects and/or operations. In some embodiments, through a simple counting of the occurrences of the releasing of the blocking mechanism, the administered dose can be calculated at any time. Since the piston advance, as described above, is predetermined on a one-to-one basis through the ratchet teeth, with knowledge of the conversion ratio a piston stroke and thus an expelled agent dose can be associated one-to-one with the displacement of the blocking means by one ratchet wheel of the ratchet teeth. Thus, through a simple multiplication of the number of occurrences by this agent dose, the total agent dose can be calculated at any time.

While it was stated above that the blocking mechanism includes a blocking teeth and a catch that works together with this teeth, in principle any suitable blocking mechanism known from the prior art can be used for the expelling device according to the present invention. For example, the holding means can be a band that is permanently blocked by a band block known from the prior art and that, upon release of the band block, slips through until the band block again clamps the band. It goes without saying that such a band block can be combined with each of the above-described blocking mechanism. In such a variant, the dose to be expelled would, as described above, be determined on a one-to-one basis through a ratchet teeth and a catch working together with this teeth. The catch could, however, be moved back and forth between the two end positions without greater forces of resistance. The actual forward turning of the blocking means would then be triggered through the release of the band block. When the catch becomes engaged again with the ratchet teeth, the further advance of the piston is ended. Subsequently, the band block again clamps the band.

It should be appreciated that the catch, e.g., the anchor escape lever or the rotatable spindle, can be locked into each of its end positions in order to prevent an uncontrolled displacement of the catch.

As described above, the expelling device according to the invention can be installed in both manually-actuated and electronically-actuated infusion or injection apparatuses. These can be used for injection of a medical agent, but also of a diagnostic agent, in human, animal, or plant tissue. Due to the low energy demand of the expelling device according to the invention, the latter is especially suitable for application in infusion pumps for the repeated release of comparatively small doses of a therapeutic agent over a comparatively long period of time.

An especially preferred use relates to the long-term release of insulin for adjustment of the blood-sugar level of diabetes patients. For this purpose, the infusion pump can be controlled by a controller that is connected to a system of sensors for determining the blood-sugar level. The minimally administrable agent dose is here substantially predetermined through the ratchet teeth of the blocking means and through the conversion ratio of the expelling device selected in each case and can be selected so as to be appropriately low. Through a simple counting of how often the blocking mechanism is released, the control electronics at all times know how large the expelled dose is. This simplifies the control expense considerably, since simple timing-control circuits can be used. Due to the low energy consumption of the expelling device, the infusion pump can be operated over an advantageously long period of time. In particular for diabetes treatment, such an infusion pump could expel the insulin through a 31-gauge needle.

In the foregoing, embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A controller for controlling the delivery of a liquid agent in doses, comprising:
a propulsion mechanism for moving a piston to deliver a dose of the agent, wherein the propulsion mechanism exerts a motive force on the piston substantially constantly unless interrupted; and
a mechanism for selectively interrupting the motive force.

2. The controller according to claim 1, wherein the controller is operably incorporated into an infusion pump.

3. The controller according to claim 2, further comprising an electronic component operably coupled to the mechanism for selectively interrupting the motive force.

4. A control device for the dosed expelling of a liquid agent, including from an infusion pump, comprising:
a receptacle for storing the liquid agent;
a propulsion mechanism for advancing a piston toward an outlet of the receptacle in order to expel the agent, wherein the propulsion mechanism continuously acts on the piston with a propulsive force; and
a blocking mechanism whereby a piston advance is blocked and, in order to expel the agent, released.

5. The control device according to claim 4, wherein the blocking mechanism is designed such that upon the release of the propulsive force the piston is advanced by a preset stroke in order to expel a preset dose of the agent.

6. The control device according to claim 4, wherein the propulsion mechanism prestresses the piston in relation to a reference point that is stationary in relation to the blocking mechanism.

7. The control device according to claim 6, wherein the propulsion mechanism includes a compression spring, one end of which acts on the piston and the other end of which is held fixed.

8. The control device according to claim 4, wherein the propulsion mechanism includes a pressurized chamber in which a gas is stored under pressure and which has a flexible chamber wall, so that the gas can expand in order to propel the piston when the blocking mechanism has been released.

9. The control device according to claim 4, wherein the blocking mechanism includes a blocking means and a catch that works together with the blocking means, which catch in a first position blocks the piston propulsion and in a second position releases the piston propulsion in order to effect the expelling of the agent.

10. The control device according to claim 9, wherein the blocking means includes teeth and the catch includes an anchor escape lever with two pawls, which lever can be swung around an axis in order to block and release the blocking means.

11. The control device according to claim 10, wherein the anchor escape lever is designed such that when the first pawl is engaged with the teeth of the blocking means, the other pawl is positioned centrally between two teeth.

12. The control device according to claim 9, wherein the propulsion mechanism comprises a toothed rack having outer teeth, and the catch cooperates with the outer teeth.

13. The control device according to claim 9, wherein the blocking means comprises teeth, and the catch works together with the teeth of the blocking means.

14. The control device according to claim 9, wherein the catch is designed as a blocking spindle with a rotatable shaft that carries two blocking projections that cooperate with the blocking means.

15. The control device according to claim 14, wherein the blocking means comprises teeth and the blocking projections are applied to the rotatable shaft in a staggered manner, so that in a first angular position of the shaft a first blocking projection engages the teeth of the blocking means and the second blocking projection is positioned centrally between two of the teeth, and so that in a second angular position of the shaft the second blocking projection engages the teeth of the blocking means and the first blocking projection is positioned between two teeth.

16. The control device according to claim 9, wherein the blocking means comprises a ratchet wheel is attached to one end of a toothed or threaded rod, the other end of the toothed or threaded rod transferring the propulsive force to the piston.

17. The control device according to claim 9, wherein the blocking means includes two sets of teeth that are offset with respect to each other, which sets work together with a blocking projection of the catch, so that in a first position of the catch one blocking projection works together with one set of teeth and in a second position of the catch the other blocking projection works together with the other set of teeth, the other blocking projection in each case releasing the corresponding teeth.

18. The control device according to claim 9, wherein, in order to trigger the expelling of the agent, the catch is displaced against a restoring force that restores the catch to an initial position.

19. The control device according to claim 4, wherein the blocking mechanism is electronically controlled.

20. The control device according to claim 4, wherein the blocking mechanism includes a mechanical actuation means for releasing the blocking of the piston.

21. The control device according to claim 4, wherein the propulsion mechanism is pre-stressed over the entire stroke of the piston.

22. The control device according to claim 4, wherein the propulsion mechanism is pre-stressed over a displacement distance that is shorter than the entire stroke of the piston, the control device further comprising a detector being provided for determining whether the propulsive force has fallen below a threshold value, and a mechanism for increasing the propulsive force of the propulsion mechanism.

23. The control device according to claim 4, wherein a holding means restrains the piston against the continuous propulsive force of the propulsion mechanism.

24. The control device according to claim 4, wherein a band restrains the piston against the continuous propulsive force of the propulsion mechanism, the blocking mechanism comprising a band block whereby the band is permanently blocked and temporarily released in order to expel the agent.

25. An infusion pump for the long-term release of an agent, comprising:
a receptacle for storing the liquid agent;
a propulsion mechanism for advancing a piston toward an outlet of the receptacle in order to expel the agent, wherein the propulsion mechanism continuously acts on the piston with a propulsive force;
a blocking mechanism for blocking and allowing the advance of the piston; and
a control apparatus that repeatedly releases the blocking mechanism in order to expel the agent in repeated, selected doses.

26. The infusion pump according to claim 25, wherein the control apparatus is electronic.

27. The infusion pump according to claim 26, wherein the control apparatus is digital.
